(19) United States
(12) Patent Application Publication
(10) Pub. No.: US 2026/0096831 A1
(43) Pub. Date: Apr. 9, 2026

(54) SYSTEMS, DEVICES, AND METHODS FOR CONTROLLING ACTUATION OF TISSUE RESECTION DEVICES

(71) Applicant: Prana Thoracic, Inc., Houston, TX (US)

(72) Inventors: Kenneth M. BUECHE, Friendswood, TX (US); Ethan FRANCK, Denver, CO (US); Jorge E. SALAZAR, Houston, TX (US); Shane KORTHUIS, Denver, CO (US); Laura K. GONZALEZ, Houston, TX (US)

(21) Appl. No.: 19/399,418

(22) Filed: Nov. 24, 2025

Related U.S. Application Data
(63) Continuation of application No. PCT/US2025/043743, filed on Aug. 27, 2025.
(60) Provisional application No. 63/687,436, filed on Aug. 27, 2024.

(51) Int. Cl. A61B 17/3207; A61B 17/32; A61B 18/00; A61B 18/14

(52) U.S. Cl. CPC A61B 17/320758; A61B 18/14; A61B 2017/320064; A61B 2018/00595; A61B 2018/141

(57) ABSTRACT

In some embodiments, an apparatus includes a handle assembly configured to be coupled to a tissue excision device. The tissue excision device can be configured to excise a target tissue site from a lung. The handle assembly can be configured to transition between a first operation and a second operation mode. The handle assembly in the first operation mode configured to form a portion of a tissue column. The handle assembly in the second operation mode configured to separate the tissue column from surrounding tissue, the handle assembly configured to transition from the first operation mode to the second operation mode after the tissue excision device forms a tissue column including the target tissue site.

### CROSS-REFERENCE TO RELATED APPLICATIONS

This application is a continuation of PCT Application No. PCT/US2025/043743, filed Aug. 27, 2025, entitled, “SYSTEMS, DEVICES, AND METHODS FOR CONTROLLING ACTUATION OF TISSUE RESECTION DEVICES,” which claims priority to and the benefit of U.S. Provisional Application No. 63/687,436, filed Aug. 27, 2024, entitled, “SYSTEMS, DEVICES, AND METHODS FOR CONTROLLING ACTUATION OF TISSUE RESECTION DEVICES,” the disclosure of which is hereby incorporated by reference in its entirety.

### TECHNICAL FIELD

Embodiments described herein relate to actuation of tissue resection or excision devices for percutaneous and minimally invasive lung surgery.

### BACKGROUND

Minimally invasive tissue excision or resection procedures are becoming more accessible as clinicians have access to improved surgical devices and training. Minimally invasive surgical resection (i.e., wedge resections, lobectomies, pneumonectomies, etc.) can reduce the barriers to providing life-changing care for patients. However, minimally invasive lung procedures present challenges because access to the site is via the chest wall, which blocks visibility of the surgical site and makes locating target tissue difficult. Additionally, access through the chest wall requires bypassing the ribs, which can apply high amounts of stress on the system. The systems, devices, and methods described herein address these challenges and provide a tissue excision system capable of minimally invasive tissue excision through the chest wall.

### SUMMARY

In some embodiments, an apparatus comprises a tissue excision device configured to resect target tissue from a lung, the tissue excision device including one or more tubular members defining an inner volume and configured to advance through lung tissue to form a tissue column within the inner volume; and a handle assembly configured to be coupled to a tissue excision device, the handle assembly configured to transition between a first operation mode in which a first actuation assembly causes the one or more tubular members to advance through the lung tissue, seal a portion of the lung tissue, and cut the portion of the lung tissue to form the tissue column, and a second operation mode in which a second actuation assembly causes a snare to separate the tissue column from surrounding tissue, the handle assembly configured to be transitioned from the first operation mode to the second operation mode after the target tissue is disposed in the inner volume of the one or more tubular members.

In some embodiments, an apparatus comprises a first actuator coupled to a helical coil of a tissue excision device, the helical coil including a first electrode on a surface thereof, the first actuator including a wheel configured to rotate a shaft coupled to the helical coil to cause the helical coil to rotate distally through tissue; a first lever coupled to a tubular member including a second electrode on a distal end thereof, the first lever when actuated configured to apply a first force to the tubular member to move the tubular member distally to clamp a portion of tissue between the first electrode and the second electrode, the first electrode and the second electrode configured to be activated with energy to seal vessels in the portion of tissue; and a second lever coupled to a cutting device, the second lever when actuated configured to apply a second force to the cutting device to cause the cutting device to advance through the portion of tissue and cut the portion of tissue.

In some embodiments, an apparatus comprises a first actuator coupled to a helical coil including a first electrode, the first actuator configured to cause the helical coil to rotate distally through tissue; a second actuator coupled to a tubular member including a second electrode on a distal end thereof, the second actuator configured to be actuated to cause the tubular member to move distally to clamp tissue between the first electrode and the second electrode, the first electrode and the second electrode configured to be activated with energy to seal vessels in the clamped tissue; a mode button configured to cause the tubular member to withdraw proximally to expose a snare near a distal end of the tubular member; and a snare actuator configured to be moved to a first position to cause the snare to move a portion of tissue toward the first electrode and the second electrode, the snare actuator configured to be moved to a second to cause the snare to sever the portion of tissue from surrounding tissue.

In some embodiments, an apparatus comprises a housing defining an inner volume; a first actuator disposed in the inner volume of the housing and coupled to a tubular member including a helical coil at a distal end thereof, the first actuator configured to cause the helical coil to rotate distally through tissue of a lung; a locking mechanism at least partially disposed in the housing, the locking mechanism in a locked configuration configured to prevent movement of the first actuator, the locking mechanism configured to transition to a locked configuration when a distal end of the helical coil is at a predetermined depth relative to a surface of the lung; and a shaft configured to extend through the inner volume of the housing such that a portion of the shaft is slidably disposed through a portion of the locking mechanism, the shaft coupled to a tissue anchor configured to anchor to a target tissue site in the lung, the shaft including a surface feature at a position along the shaft and configured to engage with the portion of the locking mechanism when the helical coil is at the predetermined depth to transition the locking mechanism to the locked configuration.

### BRIEF DESCRIPTION OF THE DRAWINGS

FIG. 1 is a schematic block diagram of a handle assembly for controlling a tissue excision device, according to an embodiment.

FIGS. 2A-2B depict a skin incision blade and a tissue dilator, respectively, according to an embodiment.

FIG. 3A shows a sleeve configured to be disposed in a port.

FIG. 3B shows a cavity sleeve catch configured to receive the sleeve of FIG. 3A and couple the sleeve to a port.

FIGS. 3C-3D show the port configured to couple to the cavity sleeve catch of FIG. 3B and receive the sleeve of FIG. 3A, according to an embodiment.

FIGS. 4A and 4C show an anchoring system including an anchor localization needle with an anchor loaded and deployed, according to an embodiment. FIG. 4B shows a preformed anchor basket, according to an embodiment.

FIG. 5A shows a wire stiffener with an anchor wire of the anchoring system of FIGS. 4A-4C disposed therethrough, according to an embodiment.

FIG. 5B shows the anchor basket seated in a distal tip of the wire stiffener, according to an embodiment.

FIG. 5C shows a notch disposed on a proximal end of the wire stiffener.

FIGS. 5D-5F show a depth lock of a handle assembly for controlling a tissue excision device configured to couple to the notch on the wire stiffener of FIGS. 5A-5C.

FIG. 6A shows a handle assembly for controlling a tissue excision device, according to an embodiment.

FIG. 6B shows the handle assembly of FIG. 6A with the left cover removed, according to an embodiment.

FIG. 6C shows a direction of movement of actuators of the handle assembly of FIGS. 6A-6B, according to embodiments.

FIGS. 7A-7C show a distal end of the tissue excision device including a first electrode, a second electrode, a cutting device, and a snare.

FIG. 8 shows a close-up view of a depth control screw coupled to a coring wheel of the handle assembly of FIGS. 6A-6C.

FIGS. 9A-9B show a distal end of the tissue excision device in a first configuration, in which a snare is residing in a groove, and a second configuration, in which a portion of the snare is visible after actuation of the snare, respectively, according to an embodiment.

FIG. 10A shows the snare of the tissue excision device of FIG. 9 pulling a tissue within the excision device toward a pair of electrodes, according to an embodiment.

FIG. 10B shows the snare severing sealed tissue within the excision device such that the tissue may be removed from a lung of a patient, according to an embodiment.

### DETAILED DESCRIPTION

Embodiments described herein generally related to systems, devices, and methods for percutaneous and minimally invasive surgery (e.g., lung surgery) for the removal of tissue for diagnosis, treatment, and other associated procedures. In some embodiments, the systems, devices, and methods described herein may be applied to other solid organs as well. Specifically, embodiments described herein cover (1) devices and methods to control depth of coring into tissue; (2) simplified designs for excision of a tissue column at the end of the coring procedure; and (3) improved general usability of systems and devices for excising tissue from a patient. Embodiments described herein may include a tissue excision device (i.e., a tissue resection device) including or coupleable to a handle assembly configured to control actuation of a distal end of the tissue excision device. The tissue excision device, when used in conjunction with accessories, may promote stabilization of the surface of the tissue to be excised (e.g., the lung) at the beginning of the procedure to improve accuracy during minimally invasive excision of tissue from solid organs. The tissue excision system described herein may provide means for shifting associated excision procedures from the operating room to an outpatient setting.

Use of minimally invasive lung procedures has increased as clinicians have access to improved surgical devices, knowledge, and training, thereby reducing the barriers and challenges associated with minimally invasive techniques and making the percutaneous route accessible for surgical lung resection (i.e., wedge resections, lobectomies, pneumonectomies, etc.). The systems, devices, and methods described herein may enable procedures in which the patient may be administered local pain management at the incision site and light, conscious sedation for overall patient management and removal of tissue of interest.

This disclosure is related to U.S. Patent Publications US 2021/0378731 entitled, “Tissue Resection Control System and Methods,” filed May 13, 2021; US 2021/0393332 entitled “Methods and Devices for Navigating a Tissue Resection Device,” filed May 13, 2021; US 2022/0047314 entitled, “Tissue Dilation and Resection Systems and Methods,” filed May 13, 2021; and US 2022/0047322 entitled, “Coring and Amputation Devices, Systems, and Methods,” filed Jun. 21, 2021, the disclosure of each of which is incorporated by reference herein in its entirety. These disclosures include information related to improving localization of tissue of interest; preparation of a patient for a percutaneous, device-compatible port; control of centering target within the port and lung stability; depth control while coring to tissue of interest; snaring of the bottom of the tissue column and finally the excision of said column of tissue utilizing the same electrode used during the coring procedure.

Embodiments herein describe a tissue excision system (hereinafter, “system”) that includes a tissue excision device (i.e., a tissue resection device) that can seal blood vessels while coring into tissue (e.g., parenchyma) by applying energy to the tissue (e.g., radiofrequency (RF) energy). The system may include an anchoring system configured to localize nodules, tumors and/or tissue of interest. In some embodiments, a method of using the system may start with deploying the anchoring system. In some embodiments, the anchoring system may include an anchor wire, a localization needle, and/or a wire stiffener (e.g., to provide compatibility with and/or couple the anchoring system to the coring system). The anchor system can be configured such that the anchor system can be left in the patient temporarily. Therefore, the anchor system may provide an operator with the ability to leave the anchor wire within the patient during transportation and associated movements. The tissue excision system (or kit) may also include a tissue dilator for preparing the intercostal space for a percutaneous port (hereinafter, “the port”). For example, the tissue dilator may be configured to form expand an opening in the chest such that the port can be disposed therethrough, The port may be configured to provide access through the chest wall and parietal pleura to the visceral pleura (i.e., the lung surface) of a patient. In some embodiments, the port can be a single lumen trocar for direct access to the lung. In some embodiments, the port can be configured for suction-induced stabilization for firmly grasping the surface of the lung. For example, the a proximal end of the port may be configured to coupled to a vacuum source such that suction can be applied to the lung through a distal end of the port to draw the lung surface against the distal end of the port and stabilize the lung surface. The stabilization on the surface of the lung may enable more accurate placement of the tissue excision device relative to a target tissue and may hold the lung in place (e.g., stationary relative to the port and tissue excision device) during the initial coring through the pleura of the lung. In some embodiments, the port may include a dilator, stylet, obturator, and/or more complex configurations to protect the port during the insertion of the port through the intercostal space while minimizing the force required for full insertion. For example, a dilator or stylet may be disposed in a lumen of the port during insertion to provide support to prevent collapse and/or compression of the lumen of the port. For example, the dilator or stylet may be rigid or solid to provide support within the lumen of the port.

In some embodiments, the tissue excision system may operate in two modes: a first mode in which the tissue excision system is configured to core into parenchyma (e.g., core-seal-cut) and a second mode in which the tissue excision system is configured to remove the core of tissue and seal the target site (e.g., snare-seal-separate). In some embodiments, the tissue excision system may be configured to transition (e.g., manually and/or automatically) between the first mode and the second mode. In some embodiments, the tissue excision system may include a handle assembly including one or more actuators. In some embodiments, transitioning the tissue excision system between the first mode and the second mode may change a configuration of the one or more actuators of the handle assembly.

In some embodiments, the handle assembly may include a first actuator configured to actuate a tissue accumulation coil (e.g., a helical coil or helical dissector). The handle assembly may include a second actuator configured to actuate a clamping device. The handle assembly may include a third actuator configured to actuate a cutting device (e.g., cutting tube, cutting element, blade, etc.). In some embodiments, the handle assembly may include a fourth actuator configured to actuate a snare (e.g., a machinal line, a wire, etc.).

In some embodiments, the tissue excision system may include a coring wheel (e.g., a linear actuator driven by a paddlewheel) to advance the tissue accumulation coil (e.g., the helical coil or helical dissector) on the distal end of the tissue excision device. At a prescribed rotation of the paddlewheel, electrodes (e.g., bipolar electrodes) disposed on the distal end of the device may clamp and seal the accumulated tissue using energy. Following sealing, the cutting device including a circular blade may be lowered to sever the sealed tissue. This piece-wise operation may be continued until the distal end of the tissue excision device reaches a desired depth in the tissue, resulting in the tissue sample and the anchor basket of the anchor system being disposed within an inner volume (i.e., a chamber) of the distal end of the tissue excision device. When the tissue sample and/or anchor basket are disposed in the inner volume of the tissue excision device, a depth indicator may be triggered, thereby notifying the operator that the tissue excision system is ready to be transitioned from the first mode of operation to the second mode of operation.

In some embodiments, when the tissue excision system is in the second mode of operation, a snare, which is disposed at a distal end of the tissue excision device is exposed. The snare can be disposed around a column of tissue, including the tissue sample, which is disposed in the distal end of the helical dissector. The snare may pull at least a portion of the tissue column (e.g., a bottom portion of the tissue column) through and/or between the electrodes. In some embodiments, at least a portion of the tissue column (e.g., a distal portion) may then be clamped and sealed by the electrodes. While the sealed tissue column is clamped, the snare may be further pulled, severing the column of tissue from a bottom of the cored cavity.

The systems, devices, and methods described herein may enhance efficacy and safety during minimally invasive and open tissue excision procedures and provide a surgical access channel, offering continuous, open access to the cored parenchyma for the introduction of other surgical devices and/or therapies for therapeutic purposes. The systems, devices, and methods described herein may offer an enhanced means for minimally invasive solutions for the excision of tissue for definitive diagnosis of cancers and/or other medical needs. Embodiments described herein address a critical need in the field of thoracic or other surgeries by facilitating more accurate and safer procedures, with the potential to significantly improve patient outcomes.

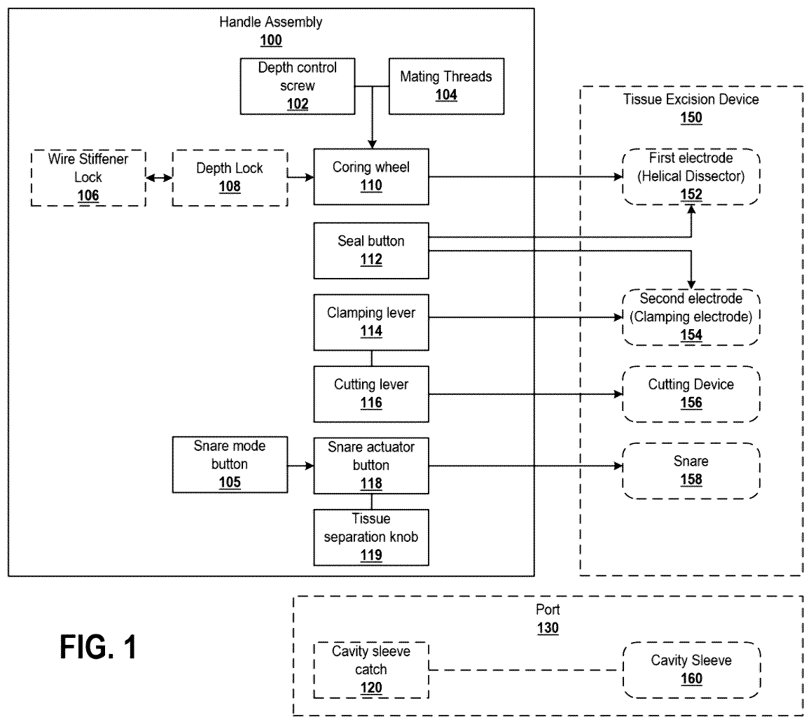

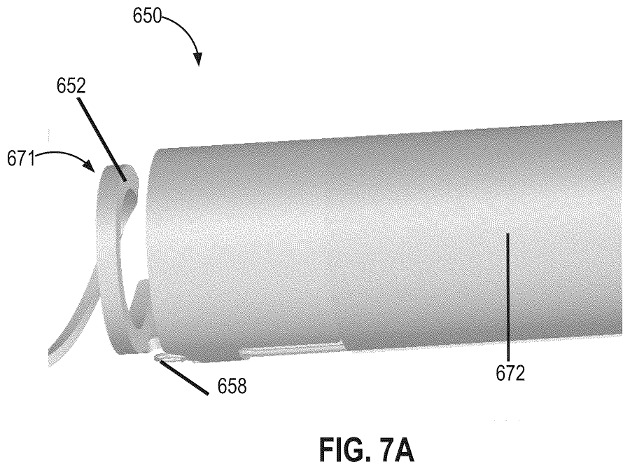

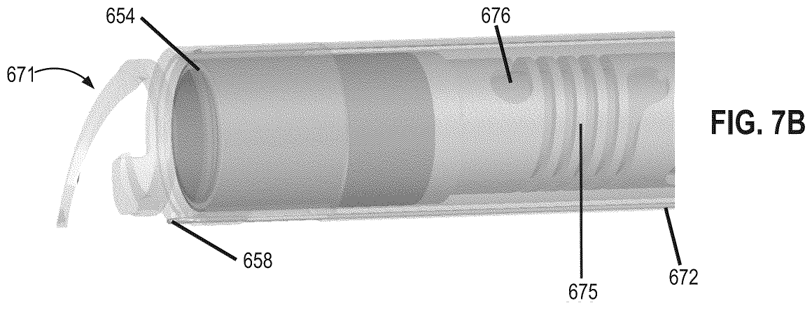

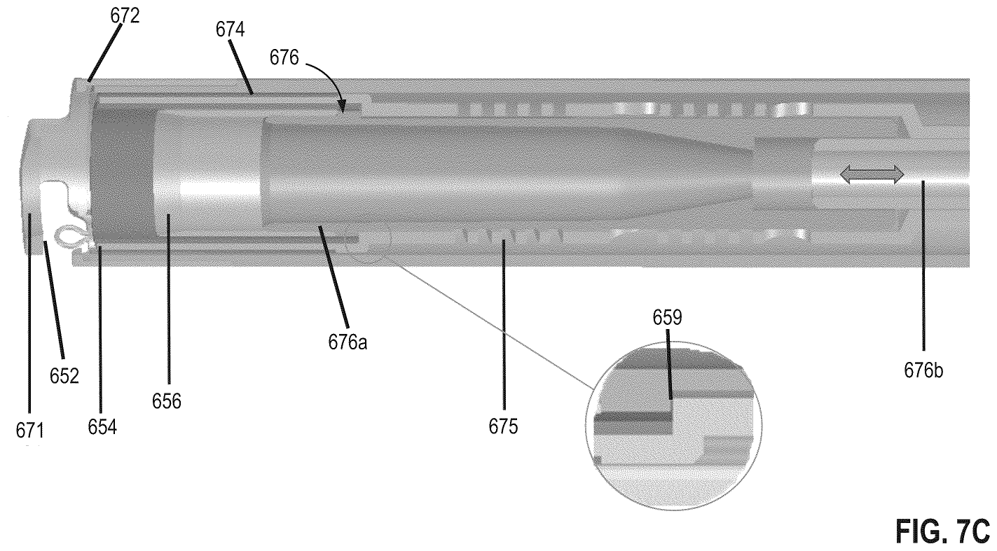

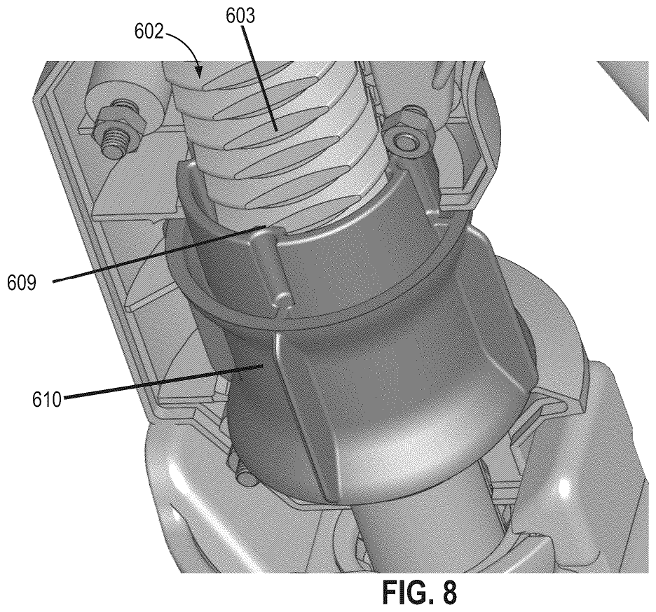

FIG. 1 is a schematic block diagram of a handle assembly 100 for controlling a tissue excision device (or tissue resection device) 150, according to an embodiment. In some embodiments, the tissue excision device 150 may include one or more tubular members. The one or more tubular members may be concentrically arranged and configured to move relative to one another. The one or more tubular members may define an inner volume and be configured to advance through the tissue to form a tissue column within the inner volume. In some embodiments, the tissue excision device 150 may include a first tubular member and a second tubular member disposed inside the first tubular member. In some embodiments, the tissue excision device may include a cutting device 156 disposed inside the second tubular member. In some embodiments, a helical dissector (e.g., a helical coil) may be coupled to a distal end of the first tubular member. The helical dissector may include a first electrode 152 disposed thereon. For example, the helical dissector may include a first electrode 152 disposed on a proximal surface thereof. A distal end of the second tubular member may include a second electrode (i.e., the clamping electrode) 154 on a distal surface thereof. In some embodiments, the second tubular member may include one or more compliant features (e.g., cut outs, springs, deformable material, coil, etc.) to enable the second tubular member to be compliant (e.g., deformable, biasing, compressible) along a longitudinal axis of the second tubular member. For example, the second tubular member may include one or more cut outs in an outer surface thereof configured to increase a compliance of the second tubular member along the longitudinal axis. In some embodiments, the tissue excision device 150 may include a third tubular member. The third tubular member may include the cutting device 156. For example, the third tubular member may include a cutting edge on a distal end thereof. In some embodiments, the tissue excision device 150 may only include a first tubular member including the helical coil and a second tubular member having a first portion including the second electrode and a second portion including the cutting device. The tissue excision device 150 may further include a snare 158. The snare 158 may be configured to gather tissue disposed in an inner volume defined by the tissue excision device 150 and/or cut a portion of the tissue disposed in the inner volume such that the tissue may be removed. Further details of the distal end of the tissue excision device 150 are described and shown in FIGS. 7A-7C.

The handle assembly 100 may be configured to control actuation of the tissue excision device 150 such that the tissue excision device 150 may collect target tissue for biopsy or other testing. The handle assembly 100 may be configured to transition between a first operation mode and a second operation mode. In some embodiments, the handle assembly 100 in the first operation mode may be configured to cause the tissue excision to form a tissue column in the tissue. For example, in the first operation mode, the handle assembly 100 may be configured to control the tissue excision device 150 to core tissue until a target portion of the tissue is disposed in the inner volume of the tissue excision device 150; seal at least a portion of the cored tissue; and/or cut a portion of the tissue (e.g., a portion of the sealed tissue) to form the tissue column. In some embodiments, the handle assembly 100 may include a first actuation assembly (e.g., a seal-cut assembly) configured to core, seal, and/or cut the tissue. In some embodiments, the handle assembly 100 in the second operation mode may be configured to separate the tissue column from surrounding tissue such that the tissue column can be removed. The handle assembly 100 may include a second actuation assembly (e.g., a snare assembly). For example, in the second operation mode, the second actuation assembly of the handle assembly 100 may be configured to control the tissue excision device 150 to snare (i.e., gather) at least a portion of the cored tissue column disposed in the inner volume of the device; to seal a portion of the tissue column (e.g., a portion of the snared tissue); and/or to separate (e.g., to sever) the cored tissue including the target portion such that the cored tissue may be removed from the patient. In some embodiments, the handle assembly may be configured to be transitioned from the first operation mode to the second operation mode after a target tissue is disposed in the inner volume of the tissue excision device (e.g., an inner volume defined by the one or more tubular members).

The handle assembly 100 may include a first actuator including one or more rotating portions. The first actuator may include a wheel (e.g., coring wheel) 110 operatively coupled to the helical dissector on which the first electrode 152 is disposed. The coring wheel 110 may be configured to control a depth and/or speed at which the helical dissector cores into tissue (e.g., lung tissue) of a patient when actuated by a user. The coring wheel 110 may be rotated by the user to move the helical dissector including the first electrode 152 distally. In some embodiments, when the coring wheel 110 is rotated, the helical dissector may rotate while moving distally into the tissue. For example, once the tissue excision device 150 is positioned on a surface of the lung, rotation of the coring wheel 110 may cause the helical dissector to puncture the visceral pleura and core into the parenchyma.

In some embodiments, the first actuator may include a rotating shaft or screw 102 (e.g., a depth control screw, a threaded elongate member, a threaded rod, etc.) In some embodiments, the coring wheel 110 may be coupled to a depth control screw 102. The coring wheel 110 may be configured to rotate the depth control screw 102. In some embodiments, the depth control screw 102 may coupled to the helical dissector and/or configured to move the helical dissector. In some embodiments, a portion of the coring wheel 110 may be configured to receive a portion of the depth control screw 102 therethrough. In some embodiments, a cross-section of portion of the coring wheel 110 may correspond to or match the cross-section of the portion of the depth control screw 102. In some embodiments, the coring wheel 110 may define a square through hole or cavity (e.g., opening, passage) along a longitudinal axis of the coring wheel 110 configured to receive the portion of the depth control screw 102 including a square cross-section therethrough. The cross-section of the depth control screw 102 may be configured such that an outer surface of the depth control screw 102 engages an inner surface of the through hole of the coring wheel 110 to control a relationship between rotation of the coring wheel 110 and descending of the helical dissector. In some embodiments, the depth control screw 102 may include threading on an outer surface thereof. The threading of the depth control screw 102 may be configured to engage mating threads 104 disposed in the handle assembly 100 (e.g., on an inner surface of a housing of the handle assembly 100). As the coring wheel 110 is rotated, the depth control screw 102 may ascend or descend via the mating threads 104 such that the depth control screw 102 moves longitudinally through the coring wheel 110.

In some embodiments, the coring wheel 110 may rotate to cause the helical dissector to core the tissue (i.e., coring phase) and then coring may be paused while at least a portion of the tissue is sealed (i.e., sealing phase) and cut (i.e., cutting phase) before coring resumes. In some embodiments, the coring wheel 110 may be configured to be rotated (e.g., by the user) a predetermined amount during the coring phase before the tissue is sealed and cut. In some embodiments, the forming the tissue column may include one or more sequences of coring, sealing, and cutting the tissue. In some embodiments, the coring wheel 110 may be configured to rotate between about 18 degrees clockwise and about 1,440 degrees clockwise during each coring phase, inclusive of all ranges and subranges therebetween. In some embodiments, the coring wheel 110 may be configured to rotate between about 270 degrees clockwise and about 450 degrees clockwise during each coring phase, inclusive of all ranges and subranges therebetween. In some embodiments, the coring wheel 110 may be rotated about 1 time to about 10 times during each coring phase to advance the helical dissector a predetermined distance during each coring phase. In some embodiments, the first actuator including the coring wheel 110 and the depth control screw 102 may be configured such that the tissue excision device moves distally the predetermined distance during each coring phase. The predetermined distance may be in a range between about 0.5 mm to about 40 mm, inclusive of all ranges and subranges therebetween. In some embodiments, rotating the coring wheel 110 about 18 degrees may result in the helical dissector of the tissue excision device moving distally about 0.5 mm and rotating the coring wheel 110 about 1440 degrees may result in the helical dissector of the tissue excision device moving distally about 40 mm. In some embodiments, the predetermined distance may be in a range between about 7.5 mm to about 12.5 mm, inclusive of all ranges and subranges therebetween. In some embodiments, the predetermined distance may be determined such that the helical dissector is advanced without tearing or damaging the tissue before sealing and cutting occurs. In some embodiments, the coring wheel 110 may include tactile and/or auditory feedback to provide the user with feedback associated with the distance a distal end of the helical dissector has traveled.

In some embodiments, the handle assembly 100 may include a second actuator including a lever (e.g., clamping lever) 114. After each coring phase (e.g., after the coring wheel 110 is rotated the predetermined amount), the user may actuate the clamping lever 114. In some embodiments, actuation of the clamping lever 114 may cause compression of tissue and vessels between the first electrode 152 (i.e., disposed on the helical dissector) and the second electrode 154 (i.e., disposed on the second tubular member). For example, the clamping lever 114 may cause the second electrode 154 to move toward the first electrode 152 to clamp the tissue between the first electrode 152 and the second electrode 154. In some embodiments, the first electrode 152 may be actuated toward the second electrode 154 and/or the first electrode 152 and the second electrode 154 may both move toward one another. In some embodiments, tissue is clamped between the first electrode 152 and the second electrode 154, the first electrode and the second electrode 154 can be configured to be activated to deliver energy to the clamped tissue to seal at least some vessels in the tissue. In some embodiments, the handle assembly 100 may include a third actuator including a lever (e.g., a cutting lever) 116 configured to actuate the cutting device 156 of the tissue excision device 150. The cutting lever 116 may advance the cutting device 156 distally to cut sealed tissue. In some embodiments, the clamping lever 114 may be operatively coupled to the cutting lever 116.

In some embodiments, a cable (or other flexible connector) and a cable conduit can be used to transfer motion between the clamping lever 114 and the cutting lever 116 to control a position of the second electrode 154 and the cutting device 156. In some embodiments, the seal-cut assembly (e.g., including at least the clamping lever 114, the cutting lever 116, the first and second electrodes 152, 154, the cable/cable conduit, and/or the cutting device 156) may be internal to the housing of the handle assembly 100 and configured to move up and down within the handle assembly 100. Therefore, the cable may be employed (e.g., rather than a stationary or rigid structure) to allow movement of the seal-cut assembly relative to the housing.

In some embodiments, the handle assembly 100 may include a fourth actuator including a button 112 (e.g., a seal button). Once tissue is clamped between the first electrode 152 and the second electrode 154, the seal button 112 may be actuated to initiate delivery of energy (e.g., radiofrequency (RF) energy) to the clamped tissue. The energy may be delivered via the first electrode 152 and the second electrode 154 using a bipolar energy delivery algorithm. In some embodiments, the algorithm may be configured to control the system to measure and/or compute the tissue impedance and to optimize energy delivery while limiting thermal damage to the surrounding tissue. Once sealing is complete (e.g., the generation of RF energy is complete), and with the clamping lever 114 still pulled, the cutting lever 116 may be actuated (e.g., pushed or pulled) to advance the cutting device 156 to sever the tissue disposed in the inner volume of the tissue excision device 150 (i.e., the tissue column) from the tissue clamped between the first and second electrodes 152, 154. In some embodiments, the clamping lever 114 may apply a first force on the second and/or third tubular members to move the second and/or third tubular members a first distance distally. In some embodiments, the cutting lever 116 may apply a second force on the second and/or third tubular members. The second force may move the third tubular member, with the cutting device 156 coupled thereto, distally to cut the tissue, while the second tubular member may be compressed along its longitudinal axis. In some embodiments, the second force may be greater than the first force.

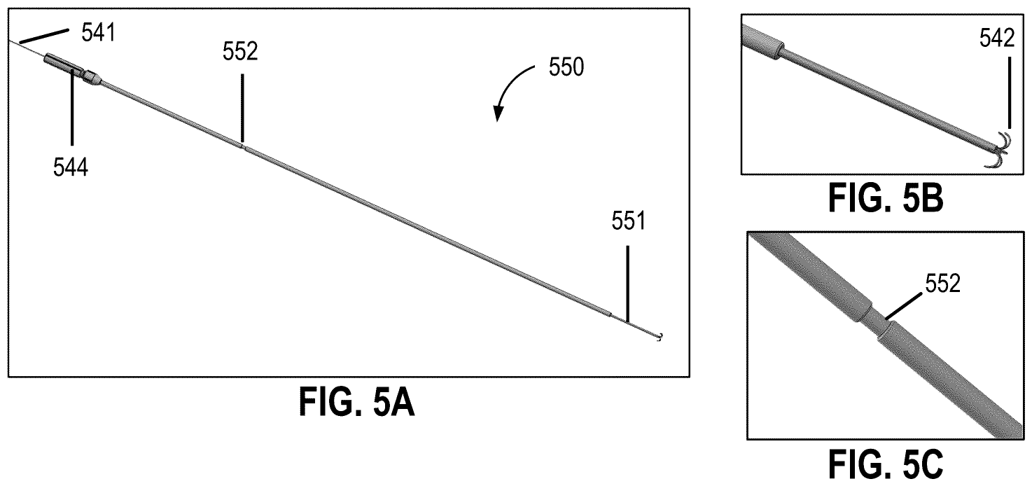

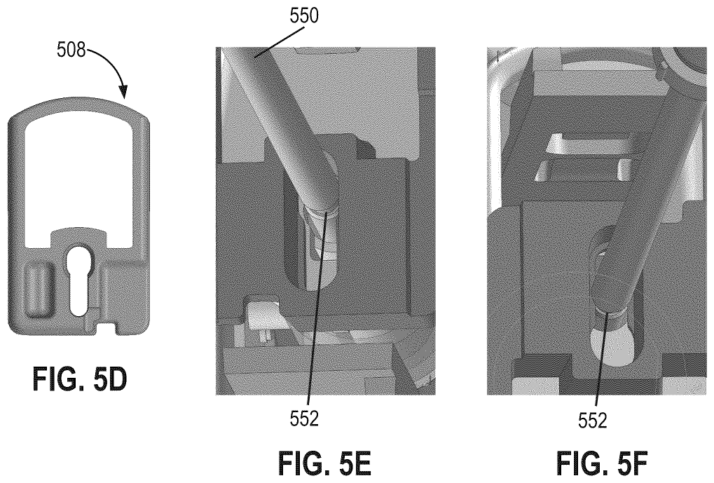

The handle assembly 100 may include a locking mechanism configured to lock the tissue excision device 150 relative to the handle assembly 100. For example, the locking mechanism may lock the tissue excision device 150 once a predetermined depth in the tissue has been reached (e.g., when a distal end of the helical dissector is a predetermined depth below a surface of the lung). In some embodiments, the locking mechanism in a locked configuration may prevent the first actuator (e.g., the coring wheel 110) from moving the helical coil. In some embodiments, the locking mechanism may include a depth lock 108 configured to control actuation of the coring wheel 110 and/or a depth to which the tissue excision device 150 can core. The locking mechanism may include a wire stiffener lock 106 configured to be coupled to or engage a wire stiffener (e.g., a shaft, an elongate member, tubular member, rod, etc.) to lock the wire stiffener relative to the housing of the handle assembly 100. In some embodiments, the wire stiffener may slidably extend through an inner volume of the housing such that a portion of the wire stiffener extends through a portion of the depth lock 108. In some embodiments, the depth lock 108 may be configured to lock the helical dissector at a depth corresponding to a depth of the target tissue site. The wire stiffener notch may be configured to engage the portion of the depth lock 108 when the helical dissector is at a predetermined depth from the surface of the lung to transition the depth lock 108 into a locked configuration. In some embodiments, the surface feature may be disposed at a predetermined position along the wire stiffener. The sequence of coring, cutting, and sealing the tissue may be repeated until a wire stiffener notch or surface feature (e.g., wire stiffener notch 552 shown in FIG. 5A) located on the wire stiffener is aligned with the depth lock 108 on the handle 100 to lock the coring wheel 110 and prevent the user from continuing to core the tissue. The depth lock 108 may include a keyhole (e.g., an opening, a through hole, a cavity, etc.) that is configured to receive the wire stiffener notch (e.g., indentation, groove, surface feature, etc.). As the thinner section of the keyhole passes over the wire stiffener notch, the depth lock 108 may be configured to engage the wire stiffener notch automatically. When the depth lock 108 is set in the notch, a tension between the wire stiffener lock 106 and the depth lock 108 may build up and prevent the helical dissector from being further descended. Therefore, the depth lock 108 may control a location of a distal end of the helical dissector to manually enable capturing target tissue in the tissue excision device. In some embodiments, after the wire stiffener notch aligns with the depth lock 108, the wire stiffener lock 106 may be unlocked (e.g., by a user and prior to actuating a snare mode button 105). Further details around engagement of the depth lock 108 and the wire stiffener notch are described in FIGS. 5A-5E below.

After the helical dissector has cored into the tissue such that the target tissue (i.e., the tissue to be tested) is disposed in the inner volume defined by the tissue excision device 150, the tissue excision system may be configured to transition into the second operating mode. In some embodiments, actuation of a mode button 105 (e.g., a snare mode button) may transition the tissue excision system from the first mode of operation to the second more of operation. Therefore, the snare mode button 105 may transition at least some of the actuators from a first configuration to a second configuration. For example, the snare mode button 105 may be actuated (e.g., pressed, twisted, pulled, touched, etc.) to initiate movement of the second electrode 154 proximally (e.g., the second electrode 154 may be raised away from the first electrode 152) to expose the snare 158. For example, the snare 158 may reside behind the second electrode 154 in a groove disposed between the distal end of the first tubular member and an internal shelf on the helical dissector. When the second electrode 154 is moved proximally, the second tubular member may unblock the groove and the snare 158 may be released or exposed. In some embodiments, the mode button 105 may be configured to cause the second tubular member to withdraw proximally to expose the snare near a distal end of the second tubular member. As the second electrode 154 moves, the cutting lever 116 may move (e.g., may automatically be moved and/or without direct user input) closer to the handle and locked into a position to prevent further use of the cutting lever 116. In some embodiments, actuating the snare mode button 105 may transition the cutting lever 116 and/or the clamping lever 114 from a first configuration in which the levers 114, 116 are a first distance from the handle 100 to a second configuration in which the levers 114, 116 are a second distance from the handle 100 smaller than the first distance. In some embodiments, the clamping lever 114 and the cutting lever 116 may be configured to move laterally towards and away from the housing. Therefore, pressing the snare mode button 105 may cause the clamping lever 114 and/or the cutting lever to be moved towards the housing. In some embodiments, the mode button 105 when actuated can be configured to lock the cutting lever to prevent the cutting device from being advanced when the handle assembly is in the second operation mode. In some embodiments, the levers 114, 116 may transition from the first configuration to the second configuration automatically in response to actuation of the snare mode button 105. In some embodiments, the cutting lever 116 in the second configuration may be locked from moving the cutting device 156. In some embodiments, the clamping lever 114 and/or the cutting lever 116 transitioning to the second configuration may expose the snare 158. In some embodiments, when the tissue excision device 150 is in the second operation mode, the cutting lever 116 may be prevented from being actuated (e.g., such that the cutting device 156 may not be advanced).

In some embodiments, the handle assembly 100 may include a snare actuator (e.g., a snare actuator button). In sone embodiments, the snare actuator button 118 may be actuated (e.g., the user may pull the snare actuator button 118 proximally) to a first position. In response to actuation of the snare actuator button 118, the snare 158 may move a portion of the tissue column disposed in the inner volume of the tissue excision device 150 (e.g., the bottom portion of the tissue column) between electrodes and position the snared tissue for sealing by the electrodes. In some embodiments, the snare 158 may form a loop or lasso at a distal end of the tissue excision device 150 around the tissue disposed inside the tissue excision device 150, and a portion of the snare 158 may extend through the tissue excision device 150 such that proximal ends of the snare 158 may be coupled to the snare actuator button 118. In some embodiments, the portion of the snare 158 may extend through a channel defined in a sidewall of the first tubular member. Actuation of the snare actuator button 118 may pull the proximal ends of the snare 158 proximally such that the loop of the snare 158 is pulled tight toward the sidewall of the first tubular member and the electrodes. In some embodiments, the electrodes may be the first electrode 152 and the second electrode 154. In some embodiments, the electrodes may be a pair of electrodes different than the first and second electrodes and configured to seal the snared tissue. Once the snared tissue is positioned between the electrodes, the clamping lever 114 may be actuated (e.g., pulled or pushed) to compress at least a portion of the tissue column between the electrodes, and the seal button 112 can be actuated to activate the generator (e.g., RF generator) to seal the at least a portion the tissue column. While the tissue is clamped and/or after the tissue is sealed, the snare actuator button 118 may be moved proximally to a second position. In some embodiments, the snare actuator button 118 is coupled to a knob 119 (e.g., a tissue separation knob 119). In some embodiments, the tissue separation knob 119 may be rotated to move the snare actuator button 118 further proximally to a second position, thereby pulling the snare 158 through the tissue residing on the outside of the electrodes (e.g., external to the internal volume defined by the tissue excision device 150) and that has been sealed with energy to sever the tissue. Once the snare 158 severs the tissue, the clamping lever 114 may be released, and the tissue excision device 150 and/or the tissue column may be moved proximally out of the patient. In some embodiments, the anchor device may be moved proximally to remove the tissue column from the patient. Removing the tissue column or the tissue core may form a tissue cavity in the tissue. In some embodiments, the devices may be disposed in the tissue cavity and/or procedures may be performed on the tissue cavity.

The system may further include a cavity sleeve catch 120 (e.g., latch, a connector, a fastener, support structure, etc.) configured to be coupled to a sleeve 160 (e.g., a cavity sleeve, a port, a channel, etc.) of the system. Post-procedure, the cavity sleeve 160 can be kept in place (e.g., maintained in the cored cavity) for a period of time to reduce blood and fluid from seeping from the tissue cavity wall. To remove the cavity sleeve 160 from the port 130 and out of the patient, the user may actuate a portion of the cavity sleeve catch 120 to release the sleeve 160. For example, the user may squeeze one or more tabs disposed on the cavity sleeve catch 120 toward one another to release the cavity sleeve 160. With the cavity sleeve 160 removed, the tissue cavity may be exposed and therapeutics may be introduced and/or a blood patch may be placed to promote aerostasis and hemostasis at the coring site.

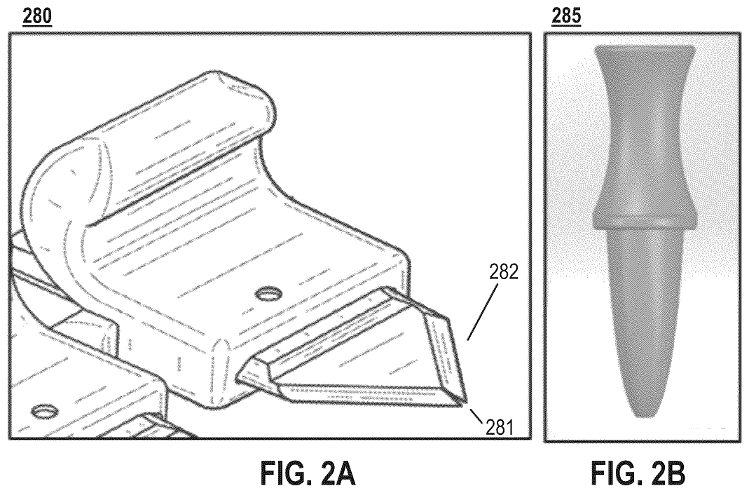

FIG. 2A depicts a skin incision blade 280 including a bore hole 281 through which an anchor wire may be disposed, according to an embodiment. The blade 282 of the skin incision blade 280 may be symmetrically designed. The skin incision blade 280 may be lowered to a skin surface of the patient and may be configured to push through the skin to form a hole that is symmetric about the anchor wire. In some embodiments, the skin incision blade 280 may form an incision having a total length to form an undersized fit relative to outer dimensions of a percutaneous port (e.g., port 130, 330). The undersized skin incision reduces vacuum leaks into the thoracic cavity and bodily fluid leakage once the percutaneous port is in place. For example, the undersized skin incision causes the skin to form a seal around the port to leaking of fluid around the edges of the port. In some embodiments, the incision can also be made by a skilled surgeon without the skin incision blade 280. FIG. 2B, is an example of a tissue dilator or rib spreader 285. The tissue dilator 285 may be configured to be disposed over the anchor wire to align the tissue dilator 285 with the incision formed by the skin incision blade 280. In some embodiments, the tissue dilator 285 can include a channel extending therethrough, the channel may terminate in a large through hole at a distal end of the tissue dilator 285. The channel may be configured to receive the anchor wire therethrough. The through hole may have a diameter larger than a diameter of the anchor wire to reduce interaction (e.g., rubbing, friction, or catching) with the anchor wire as the operator inserts the tissue dilator 285 through the intercostal space, and between the adjacent ribs.

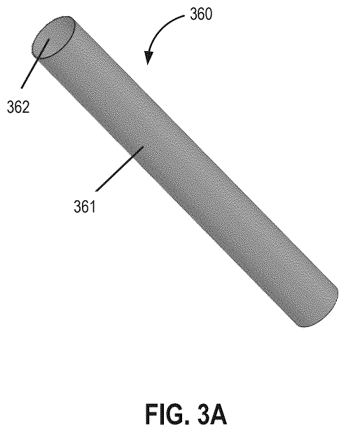

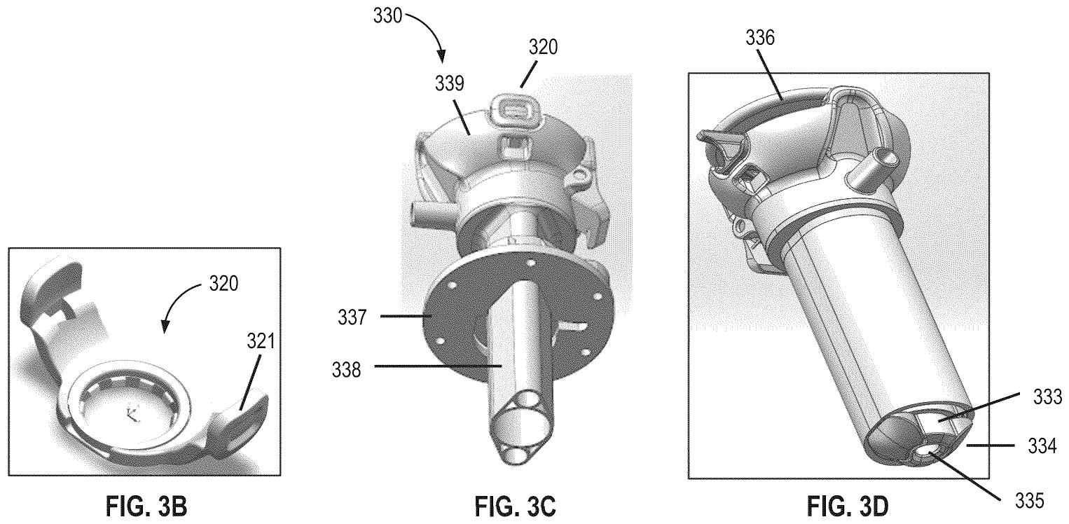

FIG. 3A illustrates a cavity sleeve 360 for covering a shaft of the tissue excision device (shaft 672 shown in FIGS. 7A-7C). The cavity sleeve 360 includes a tubular body 361 defining a lumen 362. The lumen 362 may be sized and configured to receive the shaft of the tissue excision device (not shown). In use, the cavity sleeve 360 can be placed over the shaft of the tissue excision device prior to insertion into the body. The tissue excision device, with the cavity sleeve 360 in place, can then be introduced through an incision (e.g., the incision formed by skin incision blade 280) and advanced to the target tissue site. After the desired procedure is completed, the tissue excision device can be withdrawn. In some embodiments, the cavity sleeve 360 can be left in place within the tissue cavity when the tissue excision device is withdrawn. In some embodiments, the cavity sleeve 360 can maintain access to the surgical site, allowing for easy introduction of surgical tools as needed.

FIG. 3B depicts a cavity sleeve catch 320 (e.g., latch, a connector, a fastener, support structure, etc.), according to an embodiment. FIGS. 3C-3D show a percutaneous port (hereinafter, “the port 330”) configured to be disposed through an incision created by the skin incision blade (not shown) toward a surface of the lung. The port 330 may incorporate features which allow suction to be applied to a distal end of the port 330, thereby fixing the surface of the lung to the port 330 and inhibiting movement of the lung surface relative to the port 330. To protect the distal edges of the port 330 during insertion through the rib cage, a dilator 333 (e.g., a 3-piece dilator) may be inserted through a central lumen of the port 330 to create ramped portions (e.g., set-ledged ramps) 334 on each side of the dilator 333 which may interact with the ribs. Therefore, as the port 330 with the dilator 333 is disposed between the ribs, the ramped portions may guide the gradually guide the ribs toward the outer edge of the port 330. Therefore, the dilator 333 may prevent deformation of the port 330 and may oppose a force exerted on the port 330 by the ribs. The dilator 333 may also help align the anchor wire with a center point of the lumen of the port 330. For example, the dilator 333 may include an opening (e.g., a through hole) 335 formed in a central component 336 of the 3-piece dilator 333. The opening 335 may have a diameter operable to reduce interaction with an anchor wire (not shown) configured to be disposed through the opening 335. For example, the opening 335 may have a diameter a predetermined amount larger than a diameter of the anchor wire to reduce interaction between the anchor wire and the dilator 333. The opening 335 may center the anchor wire along a longitudinal axis of the port 330. A proximal end of the port 330 may be configured for compatibility with the cavity sleeve catch 320. In some embodiments, a flange 337 may be coupled to a distal cannula 338 of the port 330. In some embodiments, the distal canula 338 can be raised and lowered relative to the flange 337, to accommodate various cavity wall thicknesses. The distal flange 337 may be lowered onto the patient's skin (corresponding to the coring site), locked relative to the port 330 and sutured to the patient's skin. In some embodiments, the distal flange 337 may rest on the patient's chest to support the port 330. This design may ensure a distal end of helical dissector of the tissue excision device is properly positioned relative to (i.e., the helical dissector aligns with) a distal end of the port 330. The helical dissector may press into and/or against the pleura about ˜2mm, to ensure all procedures are followed consistently at the beginning of the procedure for better outcomes.

The cavity sleeve catch 320 may be configured to engage (e.g., snap into, connect to, interlock with, be disposed around or within) the proximal portion 339 of the port 330. For example, the cavity sleeve catch 320 may include a spherical cross-section that corresponds to a shape of the port 330. The cavity sleeve catch 320 may engage the port 330 to allow the cavity sleeve 360 to descend through the port 330, but also prevent the cavity sleeve 360 from migrating proximally during the procedure and after the tissue excision device has been removed. In some embodiments, the cavity sleeve catch 320 may be coupled to the port 330 via a spring-actuated mechanism. Post-procedure, the cavity sleeve 360 may be kept in place, for example, a distal end of the cavity sleeve 360 may be disposed at or near a bottom of the cored tissue cavity, at the clinician's discretion. The cavity sleeve 360 can be kept in place for a period of time to reduce an amount of blood and other fluids from seeping from a wall of the tissue cored tissue. Removal of the cavity sleeve 360 may expose the cored tissue cavity for further observation and/or therapeutic reasons. To remove the cavity sleeve 360 without removal of the port 330, the operator may squeeze the tabs 321 of the cavity sleeve catch 320 toward each other and remove the cavity sleeve catch 320 and the cavity sleeve 360.

In some embodiments, the cavity sleeve catch 320 and the cavity sleeve 360 may be structurally and/or functionally similar to the cavity sleeve catch 120 and the cavity sleeve 160 described in FIG. 1, and therefore, certain details of the cavity sleeve catch 320 and the cavity sleeve 360 are not described herein again in FIG. 3A-3C.

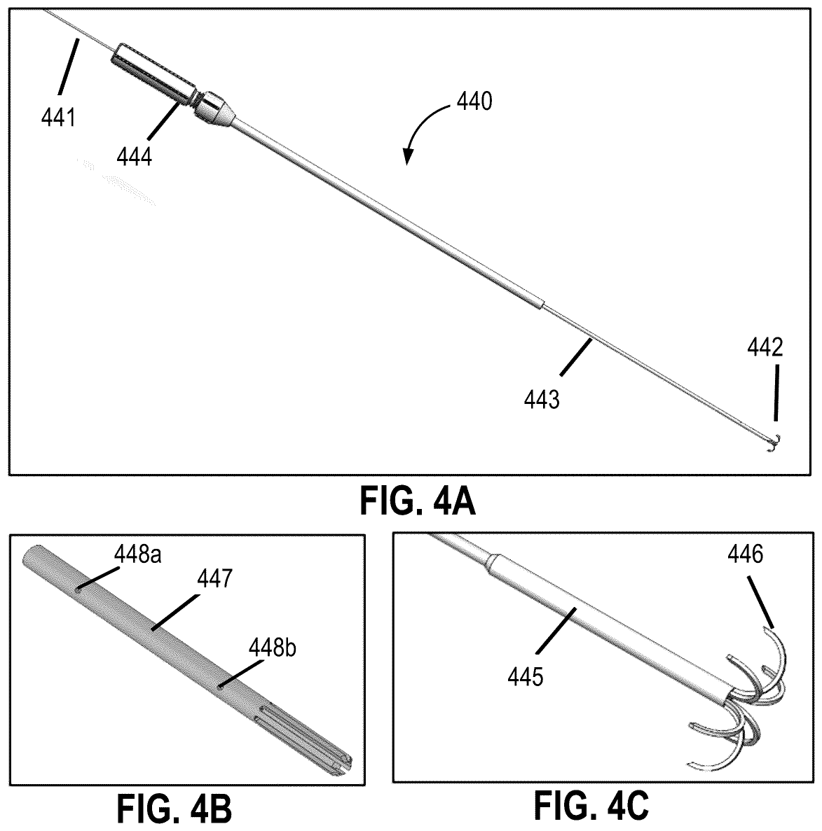

FIGS. 4A-4B depicts a localization needle 440 with an anchor wire 441 loaded therein and a distal end of the anchor wire (i.e., “the anchor basket 442”) being in a deployed configuration. The localization needle 440 may define a lumen through which a localization needle cannula 443 and the anchor wire 441 may be disposed. A wire lock 444 may be coupled to the localization needle 440 to control deployment of the anchor basket 442. In some embodiments, the wire lock 444, which may be located near a proximal end of the localization needle 440. The wire lock 444 may be preloaded, located, and/or locked on the anchor wire 441 during manufacture such that out of the box, the operator can know that the anchor basket 442 is fully and safely retracted within the distal end of the localization needle cannula 443 and that upon full distal movement of the wire lock 444, the anchor basket 442 has been fully deployed from the distal end of the localization needle cannula 443. The localization needle cannula 443 may include a hypo-tube defining a lumen through which the anchor wire 441 may be disposed. A distal end of the localization needle cannula 443 may include a pointed tip (e.g., a sharp tip, a needle tip, a puncture member) such that the distal end may puncture through thoracic wall. For example, the localization needle cannula 443 may include hypo-tubing (e.g., 18-guage hypo-tubing), and include a beveled needle tip formed on the distal end of the hypo-tubing and having a predetermined size. The needle tip having a predetermined or standard size may provide a standard size and approach to puncturing through the thoracic wall, the lung parenchyma, and localizing and passing through tissue of interest. In some embodiments, after the localization needle cannula 443 penetrates the tissue and reaches the target tissue, the anchor basket 446 may be deployed from the distal end of the localization needle cannula 443. The localization needle 440 may be removed (e.g., pulled proximally) from the patient such that just the anchor wire 441 remains in the patient with the anchor basket 442 anchored to a portion of the target tissue. In some embodiments, the skin incision may be formed (e.g., by incision blade 280) after the anchor wire 441 is disposed in the patient and the anchor basket 442 has engaged the portion of target tissue. In some embodiments, a dilator (e.g., dilator 285) may be slid over the anchor wire 441 and into the incision to prepare the incision for the port and/or tissue excision device.

In some embodiments, the localization needle 440, with the anchor wire 441 loaded, may include a radiopaque maker (not shown) embedded within a distal portion of the anchor wire 441. For example, the distal portion of the anchor wire 441 may include a tubular section 445 that is disposed proximal to the anchor basket 442. The tubular section 445 may include the radiopaque marker at a distal end of the tubular section 445. In some embodiments, the anchor wire 441 may include a tube 447, a marker, and/or a wire. The anchor basket 442 may be formed by cutting a plurality of longitudinal slots (e.g., symmetrically cutting), e.g., via a laser, in the wall of the tube 447 (e.g., a Nitinol tube), forming precursors to anchor tines 446, as shown in FIG. 4B. In some embodiments, 2 slots to 10 slots may be cut in the wall of the tube 447. In some embodiments, 6 slots may be cut in the tube 447 to form precursors to anchor tines 446. In some embodiments, the tube 447 may have an outer diameter between about 0.03 inches (in) and 0.06 in, inclusive of all ranges and subranges therebetween. In some embodiments, the tube 447 may have an inner diameter of between about 0.01 in and about 0.03 in, inclusive of all ranges and subranges therebetween. Using a proprietary tooling, the anchor tines 446 can be shape-set into the curved-back (umbrella) shape, as shown in FIG. 4C. In some embodiments, one or more holes (e.g., two holes) 448a, 448b may be laser cut into a solid portion of the tube 447, and the wire (e.g., a Nitinol wire) and the marker(s) (e.g., Platinum-Iridium marker) may be welded in place relative to the tube 447. For example, the wire may be welded to a proximal end of the tube 447 at the proximal hole 448a such that the tube 447 fits over at least a portion of the wire, and the marker may be welded on the tube 447 at the distal hole 448b. Prior to welding the wire and the marker, the distal end portion of the wire (e.g., a 4 in. region on the distal end the Nitinol wire) may be heat treated to shift an Austenitic finish (A_{f}) temperature of the wire to above ˜104° F. (˜40° C.) leaving the distal end of the wire in a martensitic state similar to a compliant (i.e., floppy) tip in a medical guidewire. The distal, compliant region may allow for easier and safer securing of the wire to the patient's skin to improve stability of the anchor basket 442 and protection of the entry wound in the skin. This design may reduce trauma and potential dislodgement of the anchor basket 442 during patient movement after deployment.

FIGS. 5A-5B shows a wire stiffener 550 disposed over the wire anchoring system of FIG. 4A. In some embodiments, the wire stiffener 550 can extend over (e.g., be placed over) the anchor wire 541 and act as a rail (e.g., a guide, a path, etc.) for the tissue excision device to follow. For example, after an incision is made centered around the anchor wire 541, and the port has been disposed in the incision, the wire stiffener 550 may be disposed over the anchor wire 541. In order to perform the tissue excision procedure, the wire stiffener 550 may be loaded into the system such that the tissue excision device (not shown) may be positioned in proximity to the previously inserted and prepared port (e.g., port 130, 330). A proximal portion of the anchor wire 541 may be loaded into the distal cannula 551 of the wire stiffener 550. In some embodiments, the wire stiffener 550 and the tissue excision device may be disposed over the anchor wire 541 simultaneously. For example, the tissue excision device may be disposed over the wire stiffener 550, and then the wire stiffener 550 and the tissue excision device may collectively be disposed over the anchor wire 541. As the anchor wire 541 is further inserted into the wire stiffener 550, the tissue excision device may be brought closer to the port and brought into alignment with the port. When the helical dissector, disposed on the distal section of the tissue excision device nears the port entrance, the proximal portion of the anchor wire 541 may move proximally through the wire stiffener 550 and emerge such that the operator can grasp the anchor wire to promote smooth docking with the port, minimizing the opportunity for dislodgement of the anchor basket relative to the tissue of interest.

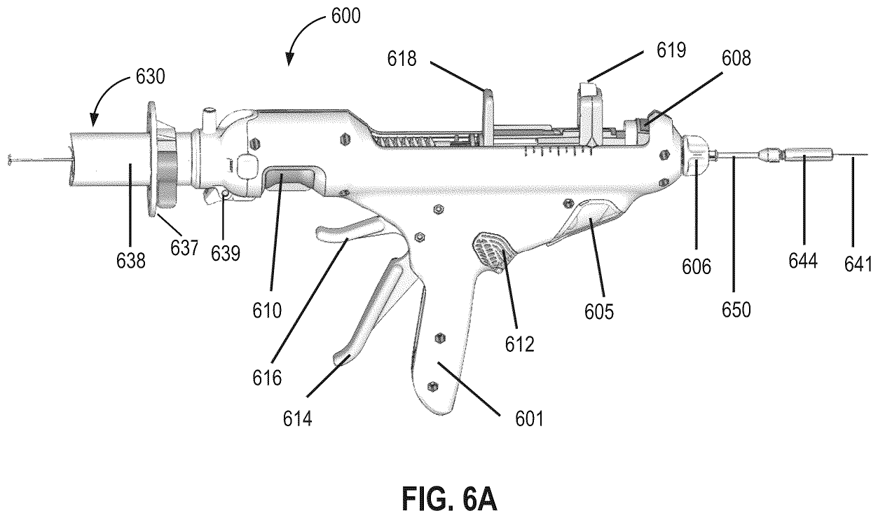

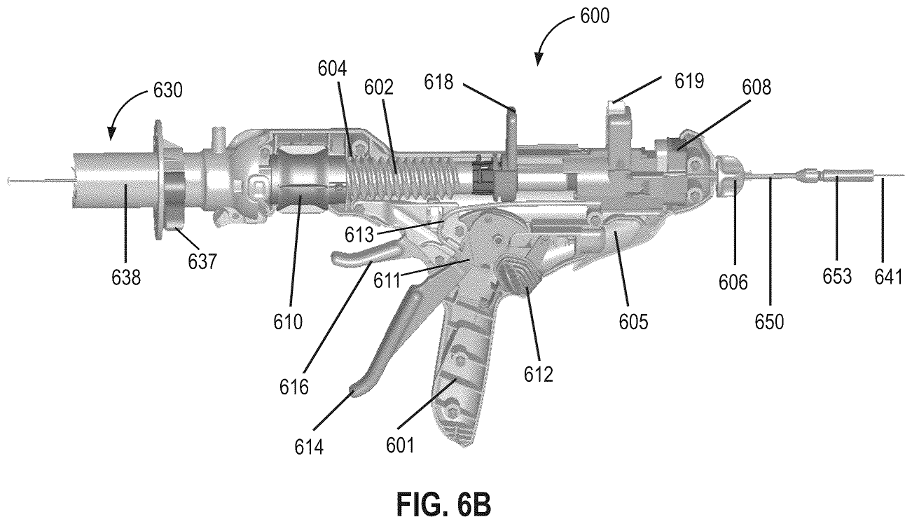

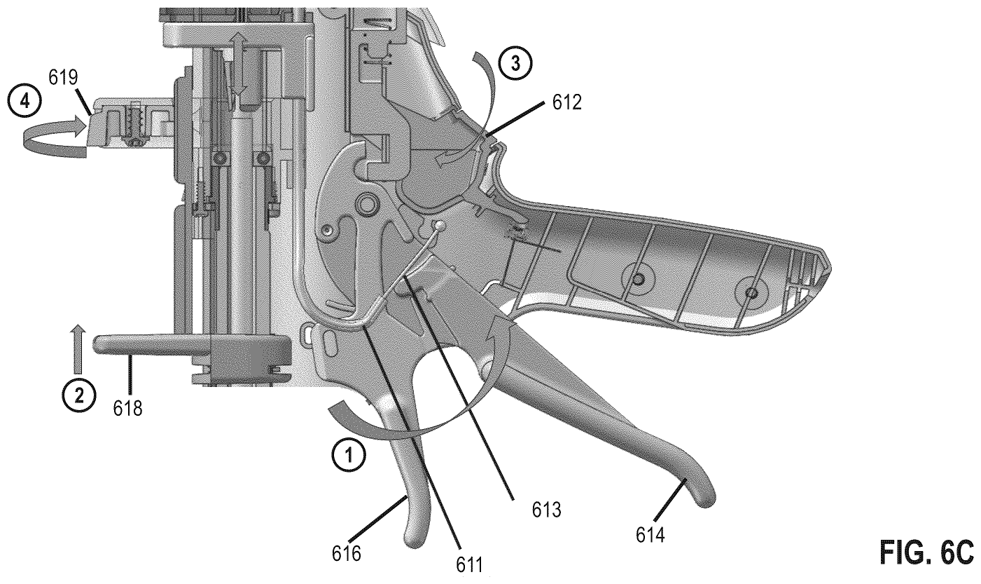

In some embodiments, the wire stiffener 550 may be disposed over the anchor wire before the tissue excision device is disposed over the wire stiffener 550. For example, the wire stiffener 550 may be disposed over the anchor wire (e.g., the proximal portion of the anchor wire 541 may be loaded into the distal cannula 551 and extend through the wire stiffener 550). After the wire stiffener 550 is locked in place relative to the anchor wire 541, the tissue excision device may be disposed over a proximal end of the wire stiffener 550 and moved distally. The tissue excision device may be attached and locked to the port via a latch (e.g., (a spring-actuated latch (such as latch 639). For example, a proximal end of the tissue excision device may couple to the port 630 as shown in FIG. 6A.

The wire stiffener 550 may be unlocked from the tissue excision device by rotating a wire stiffener lock (e.g., wire stiffener lock 606 shown in FIG. 6A) counterclockwise. After the wire stiffener 550 is unlocked from the tissue excision device, the wire stiffener 550 can be brought into position relative to the anchor basket 542 and the tissue excision device. The operator may use the proximal end of the handle of the tissue excision system to steady their hand, and grasp a proximal end (e.g., the tail) of the anchor wire 541, and move the wire stiffener 550 distally until the wire stiffener 550 comes into contact with the anchor basket 542 such that the anchor basket 542 sits in (e.g., abuts, contacts, couples to, engages, etc.) a distal end of the wire stiffener 550, as shown in FIG. 5B. In some embodiments, a proximal end of a tubular section (e.g., tubular section 445 in FIG. 4C) may engage the distal end of the wire stiffener 550 to lock the wire stiffener 550 relative to the anchor basket 542. In some embodiments, the anchor basket 542 (and/or the tubular section) engaging the distal end can create a tactile sensation that can be felt by the operator during the process. With the anchor basket 542 seated against the wire stiffener 550, the operator can lock the wire stiffener 550 in place relative to the handle assembly by rotating the wire stiffener lock clockwise until tight. The anchor wire lock 544 can be slid over the anchor wire 541 and brought into contact with the wire stiffener 550, the anchor wire 541 can then be gently pulled taut, and the anchor wire lock 544 can be locked such that a position of the wire stiffener 550 relative to the anchor wire 541 is locked and the system is ready to core.

The wire stiffener 550 may be a tubular structure that may set a positional relationship between the anchor basket 542 and a notch 552 machined into a proximal region of the wire stiffener 550. FIG. 5C shows a close-up view of the notch 552 of the wire stiffener 550. For example, the wire stiffener 550 may be configured to maintain a predetermined distance between the notch 552 and the anchor basket 542. The notch 552 can serve as a location identification feature. In some embodiments, the notch 552 can trigger a locking mechanism within the tissue excision device to prevent further coring (e.g., further movement of the tissue excision device distally relative to the wire stiffener 550). FIG. 5D shows the locking mechanism (e.g., the depth lock) 508 on the handle assembly configured to engage the wire stiffener 550. As shown in FIGS. 5E-5F, the locking mechanism (e.g., the depth lock) 508 may define a through hole configured to receive the wire stiffener 550. As the notch 552 moves through the portion of the depth lock 508, the depth lock 508 may engage the wire stiffener 550 and prevent further movement of the tissue excision device distally relative to the wire stiffener 550. Further, when the locking mechanism (e.g., the depth lock) 508 is triggered, a small indicator (e.g., a flag, light, protrusion, etc.) can be extended to act as a visual notification to the operator that the wire stiffener 550 is locked in place relative to the handle assembly. As with the localization needle, the wire lock 544 may be used to hold the anchor basket 542 tight (e.g., flush, stable, secure) against the distal portion of the wire stiffener 550.

In some embodiments, the wire stiffener lock 544 and the depth lock 508 may be structurally and/or functionally similar to the wire stiffener lock 106 and the depth lock 108 described in FIG. 1, and therefore certain details of the wire stiffener lock 544 and the depth lock 508 are not described herein again with respect to FIGS. 5A-5F.

FIG. 6A depicts a handle assembly 600 coupled to a port 630 and having an anchor wire 641 and wire stiffener 650 disposed therethrough. FIG. 6B depicts the handle assembly 600 with a left cover of the handle assembly 600 removed. The anchor wire 641 and wire stiffener 650 may be locked in place relative to one another via the anchor wire lock 644. The handle assembly 600 may further include a wire stiffener lock 606 configured to control a relative position of the handle assembly 600 relative to the wire stiffener. The anchor wire lock 644 and/or the wire stiffener lock 606 may promote coring of the tissue excision device to a predetermined distance such that the distal end of the tissue excision device reaches the target tissue. As shown, a distal end of the handle assembly 600 may couple to a port 630 including a distal cannula 638 and a flange 637. In some embodiments, the handle assembly 600 can be a two-mode assembly. The first operation mode can be for coring, cauterizing/sealing, and cutting tissue, whereas the second operation mode can be for snaring, cauterizing/sealing, and separating the cored tissue sample at the bottom of the tissue column obtained during the first operation mode. In some embodiments, certain aspects of the handle assembly 600 may be structurally and/or functionally similar to the handle assembly 100, and therefore details of the handle assembly 600 are not described herein again with respect to FIGS. 6A-6C.

The handle assembly 600 may include a housing 601 defining an inner volume in which each component of the handle assembly 600 may be at least partially disposed. The handle assembly 600 may include a coring wheel 610. In some embodiments, a rotation of the coring wheel 610 can cause rotation of a depth control screw 602, and can allow lateral translation of the depth control screw 602 (or depth control sleeve) relative to the coring wheel 610. In other words, the depth control screw 602 moves along a longitudinal axis of the handle assembly 600 while the coring wheel 610 rotates about the longitudinal axis of the handle assembly 600. The depth control screw 602 outer thread is coupled to mating threads 604 in the handle housing 601, which causes the depth control screw 602 (and the coring assembly) to move up and down relative to the handle housing 601.

The coring wheel 610 may be configured to engage a shaft or first tubular member of the tissue excision device 650 to actuate a distal end of the tissue excision device 650. The distal end of the tissue excision device 650 is shown in FIGS. 7A-7C. The first tubular member may include an end effector. As shown, the end effector may include a helical dissector 671 on which a first electrode 652 can be disposed and a second electrode (e.g., the clamping electrode) 654 can be configured to contact. The distal end of the tissue excision device 650 further includes the snare 658. As shown in FIGS. 7B-7C, the distal end of the tissue excision device 650 includes the first tubular member 672 including the helical dissector 671 disposed at a distal end thereof. The first tubular member 672 defines an inner volume in which a second tubular member 674 is disposed. The second tubular member 674 includes the second electrode 654 disposed at a distal end thereof. The second tubular member 674 further includes one or more compliant features 675 (e.g., cut outs, spring, deformable material, coil, etc.) to enable the second tubular member 674 to be compliant (e.g., deformable, biasing, compressible) along its longitudinal axis. As shown, the second tubular member 674 includes one or more cut outs in an outer surface thereof and in a predetermined pattern such that the second tubular member 674 has a predetermined compliance along its longitudinal axis. The second tubular member 674 may define an inner volume in which a third tubular member 676 may be disposed. The third tubular member 676 may include a cutting device 656 on a distal end thereof. In some embodiments, the third tubular member 676 may include a first portion 676a and a second portion 676b proximal to the first portion 676a. The first portion 676a may have a first diameter, and the second portion 676b may have a second diameter smaller than the first diameter. In some embodiments, the first portion 676a may define an engagement member 659 (e.g., ledge, a ridge, a protrusion, an arm) configured to engage the second tubular member 674 when the third tubular member 676 is actuated proximally.

Referring back to FIGS. 6A-6C, the handle assembly 600 may further include a clamping lever 614 coupled to the second electrode 654 (e.g., via the second tubular member 674) and operatively coupled to a cutting lever 616. The cutting lever 616 may be configured to move the cutting device 656 (shown in FIGS. 7B-7C) of the tissue excision device 650. The handle assembly 600 may include a snare actuator button 618 configured to manipulate the snare 658 (shown in FIG. 7A) of the tissue excision device 650. The snare may further be manipulated by a tissue separation knob 619 to actuate the snare 658 to sever a portion of a tissue sample. The handle assembly 600 may include a snare mode button 605 configured to transition the handle assembly 600 between the first operation mode and the second operation mode, further details with respect to the first and second operation modes described below.

### First Operation Mode: Core-Seal-Cut

When the tissue excision device 650 is docked on the port 630, a distal tip of the helical dissector 671 may extend proud (e.g., a small portion of the distal end of the helical dissector 671 may extend proud) from the distal edge of the port 630 such that the distal tip of the helical dissector 671 pushes into the lung's surface. The user may then operate the system to begin a first coring sequence. Since at the start of the first coring sequence, no tissue has accumulated in between the first electrode 652 and the second electrode 654, the operator may rotate the coring wheel 610 5/4 of a turn (i.e., 450 degrees) clockwise. The coring wheel 610 rotating more than 360 degrees allows the helical dissector 671 to puncture through the visceral pleura and into the parenchyma of the lung. The handle assembly 600 may include a seal-cut assembly including the clamping lever 614, the cutting lever 616, one or more cables and/or conduits, and the second electrode 654 and/or the cutting device 656.

Once the rotation of the coring wheel 610 is completed, the sealing phase may begin. The operator may squeeze the clamping lever 614, which compresses the tissue and vessels between the second electrode (e.g., the clamping electrode(s)) 654 and the first electrode (helical electrode(s)) 652. In some embodiments, a cable 613 and a cable conduit 611 may be used to transfer motion between the clamping lever 614 and the cutting lever 616 to the third tubular member 676 (e.g., the second portion 676b of the third tubular member 676), which controls a position of the second electrode 654 and the cutting blade 656 (e.g., the circular cutting blade). For example, the second portion 676b of the third tubular member 676 may control motion of the first portion 676a of the third tubular member 676 and motion of the second tubular member 674. Therefore, the third tubular member 676 and the second tubular member 674 may be connected at a proximal end. In some embodiments, an actuator may be configured to apply one or more forces to the proximal end such that the one or more forces are applied to the second tubular member 674 and the third tubular member 676 simultaneously. For example, when the clamping lever 614 is actuated to deliver a first force, the third tubular member 676 may move the second tubular member 674 distally towards a flat surface of the helical dissector 671 on which the first electrode 652 is disposed to clamp tissue between the first electrode 652 and the second electrode 654. The compliant feature(s) 675 (e.g., a spring) in the shaft of the second tubular member 674 may absorb at least a portion of force transferred by the clamping lever 614 and cutter lever 616 to the second tubular member 674. The compliant feature(s) 675 may allow the cutting device 656 on the distal end of the third tubular member 676 to move distally to meet the first electrode 652 once a second force produced by squeezing the cutting lever 616 is larger than a spring force of the compliant feature(s) 675.

The cable 613 (or other flexible means) may be used because the seal-cut assembly (including the clamping lever 614, the cutting lever 616, the cables 613, conduits 611, the second electrode 654, and/or the cutting device 656) is internal to the housing 601 of the handle assembly 600 and can move up and down (i.e., proximal and distal, respectively) within the housing 601 of the handle assembly 600. The use of the cable 613 allows for simplicity for the operator and reduces the form factor of the device compared to other mechanisms such as gears. The compliant feature(s) 675 disposed on the second tubular member 674 may create a simpler, more compact biaxial design instead of a triaxial design that would use additional coaxial tubes and actuation mechanisms to move the second electrode 654 and circular cutting blade 676.

With the tissue clamped, a seal button 612 may be pressed to initiate delivery of energy to at least one of the first electrode 652 or the second electrode 654. For example, the seal button 612 may initiate delivery of RF energy via an advanced, bipolar energy delivery algorithm. The algorithm may be designed to measure and/or compute tissue impedance (e.g., near the helical dissector 671) to optimize energy delivery and limit thermal damage to the surrounding tissue. Once the RF generator is finished, and with the clamping lever 614 still pulled, the cutting phase may begin. The cutting lever 616 may be pulled to advance the third tubular member 676 including the cutting device 656 (e.g., the circular blade) to sever a tissue column of the tissue clamped between the electrodes 652, 654. In some embodiments, the cutting lever 616 may be pulled such that the second force (e.g., greater than the spring force of the compliant feature(s) 675) is applied to the third tubular member 676 to move a distal edge of the cutting device 656 towards and/or in plane with the first electrode 652. The above steps complete the first core, seal, cut sequence. The core, seal, cut sequence may be then repeated using a ¾ turn (i.e., 270 degree) counterclockwise of the helical dissector 671 until the wire stiffener notch (e.g., wire stiffener notch 552 in FIGS. 5A, 5C, and 5E-5F) is aligned with a depth lock 608 to trigger the depth lock 608 and prevent the operator from continuing to core. With the depth lock 608 set in the wire stiffener notch, tension between the wire stiffener lock 606 and the depth lock 608 prevents the operator from further descending the helical dissector 671.

FIG. 8 shows a close-up view of the depth control screw 602, according to embodiments. The relationship between rotation and descending of the helical dissector may be accomplished with a square cross-section 603 of the depth control screw which can interface with a square-shaped element 609 in an internal bore of the coring wheel 610, as shown in FIG. 8. When the coring wheel 610 is rotated, the square element 609 may engage with the square cross-section 603 of the depth control screw 602 such that rotational force applied to the coring wheel 610 may be directly transferred to the depth control screw 602. The depth control screw 602 may also interface with mating internal threads (not shown) molded into the housing halves. As the coring wheel 610 is rotated, the depth control screw 602 may rotate and ascend and/or descend via the mating thread and move longitudinally through the internal bore of the coring wheel 610.

The sequence of coring, sealing, and cutting may be continued until the distal end of the tissue excision device reaches a desired depth in the tissue, and at least one of a tissue sample and the anchor basket is disposed within an inner volume (i.e., a chamber) of the coring device at which time a depth indicator may be triggered, thereby notifying the operator that the tissue excision device is ready for the second mode of operation. In some embodiments, the wire stiffener lock 606 may be unlocked (e.g., manually) prior to entering the second mode of operation. For example, during the second mode of operation, at least a portion of the handle assembly 600 may move proximally, and therefore, the wire stiffener lock 606 may be unlocked to allow the proximal movement of the handle assembly 600 relative to the wire stiffener 650.

### Second Operation Mode: Snare-Seal-Separate

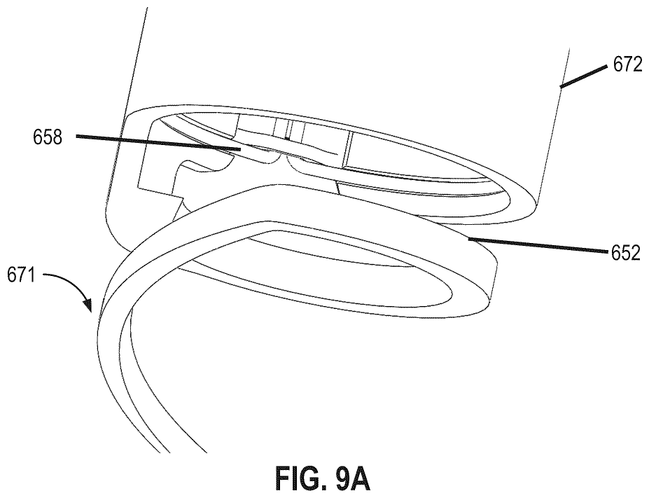

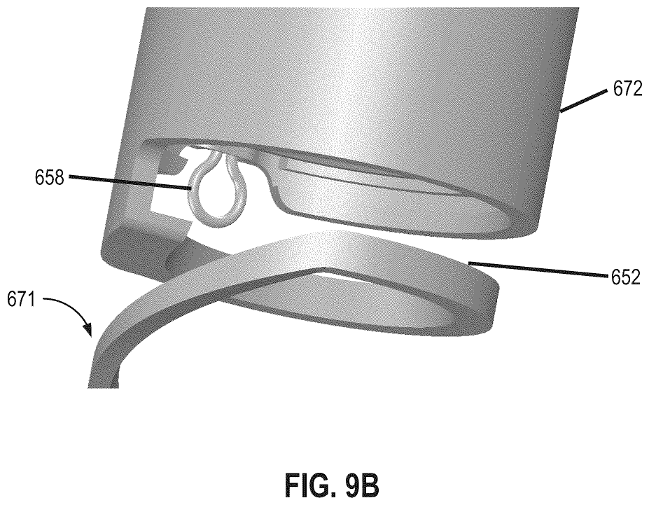

The second mode of operation is engaged upon pressing the snare mode button 605, shown in FIGS. 6A-6B. As shown in FIG. 9A, before pressing the snare mode button, the second electrode 654 covers the snare 658. Pressing the snare mode button may move the second electrode 654 proximally, exposing the snare 658 that resides behind the second electrode 654 within a groove between the distal end of the first tubular member and an internal shelf of the helical dissector 671, as shown in FIG. 9B. A ledge 659 (e.g., protrusion, ridge, arm, etc.) in the third tubular member 657 shown in FIG. 7C may pull on the second electrode 654 to forcibly retract the second tubular member 674, and therefore, the second electrode 654 when the snare mode button 605 is pressed. A mechanism to retract the second electrode 654 solves for issues of the second electrode 654 failing to retract proximally, leading to a failure to use the snare 658. FIG. 6C shows a direction of motion of actuators in the handle assembly 600 during steps of the second operation mode. When the second electrode 654 moves proximally, the cutting lever 616 may be pulled closer to the handle assembly 600 and locked into position to prevent further use of the cutting lever 616, as shown as step 1 in FIG. 6C.

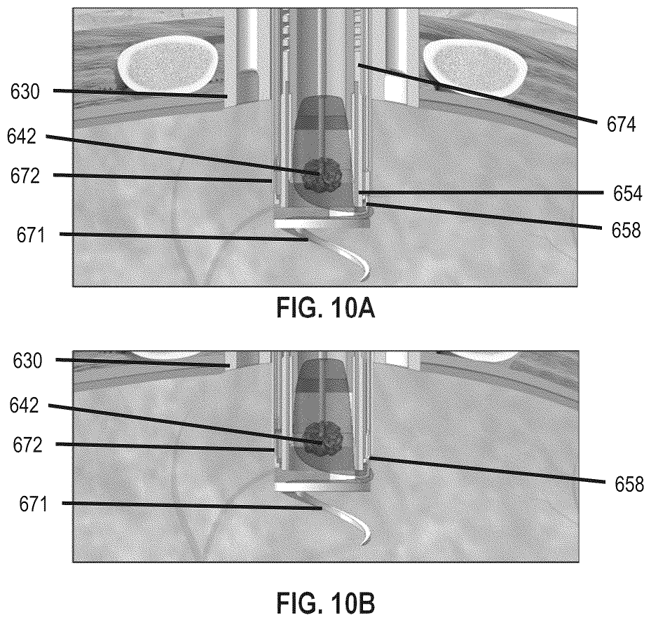

Once the snare 658 is released (e.g., exposed), the operator may raise the snare actuator button 618 (e.g., proximally) to a first position/stop, shown as step 2 in FIG. 6C. This step causes the snare 658 to pull the snared tissue column between the electrodes 652, 654 and positions the tissue for sealing, as shown in FIG. 9B and FIG. 10A. FIG. 9B depicts the snare 658 position after the snare actuator has been raised to the first position/stop without showing the tissue for clarity, whereas FIG. 10A depicts the snare position at the first position/stop of the snare actuator button with the tissue column shown.

Once a portion of the tissue is disposed between the electrodes the clamping lever 614 may be actuated to compress the tissue column between the electrodes and the seal button 612 may be actuated, shown as step 3 in FIG. 6C, to activate the RF generator. This operation seals the stem of the tissue column connecting the tissue column to the coring site. While still clamping the tissue, the operator may rotate a tissue separation knob 619, shown as step 4 in FIG. 6C, and pull the snare actuator button 618 further up (e.g., proximally) to a second position/stop, thereby pulling the snare 658 through the tissue (e.g., that resides on the outside of the electrodes 652, 654 and that has been sealed with RF energy) to sever the tissue, as shown in FIG. 10B. After severing the tissue, the clamping lever 614 can now be released, thereby completing tissue excision of the tissue column including the target tissue. To remove the tissue excision device, the coring wheel 610 may be rotated one full turn counterclockwise, and the latch (e.g., latch 321) holding the tissue excision device to the port 630 may be released to pull the tissue excision device out of the port 630. With the cavity sleeve catch (not shown) still in place the cavity sleeve (not shown) can remain in place after removal of the device.

### Cavity Sleeve and Tissue Access

After the tissue has been resected, the tissue excision device may be removed from the patient via the port 630. In some embodiments, post-procedure, the cavity sleeve can be kept in place in the cored tissue cavity for a period of time to reduce the amount of blood and other fluids from seeping from the tissue cavity wall. Removal of the cavity sleeve may expose the cored tissue cavity for further observation and/or therapeutic reasons. To remove the cavity sleeve without removal of the port 630, the operator may squeeze the tabs (e.g., tabs 321 shown in FIG. 3) of the cavity sleeve catch toward each other and remove the cavity sleeve catch and the cavity sleeve. With the cavity sleeve removed, the newly cored tissue may be exposed and therapeutics or and/or an autologous blood patch may be placed in the cored tissue cavity to promote aerostasis and/or hemostasis.

It should be understood that the disclosed embodiments are not representative of all claimed embodiments. As such, certain aspects of the disclosure have not been discussed herein. That alternate embodiments may not have been presented for a specific portion of the innovations or that further undescribed alternate embodiments may be available for a portion is not to be considered a disclaimer of those alternate embodiments. Thus, it is to be understood that other embodiments can be utilized, and functional, logical, operational, organizational, structural and/or topological modifications may be made without departing from the scope of the disclosure. As such, all examples and/or embodiments are deemed to be non-limiting throughout this disclosure Some embodiments described herein relate to methods. It should be understood that such methods can be computer implemented methods (e.g., instructions stored in memory and executed on processors). Where methods described above indicate certain events occurring in certain order, the ordering of certain events can be modified. Additionally, certain of the events can be performed repeatedly, concurrently in a parallel process when possible, as well as performed sequentially as described above. Furthermore, certain embodiments can omit one or more described events.

As used in this specification and/or any claims included herein the singular forms “a,” “an,” and “the” include plural referents unless the context clearly dictates otherwise. Thus, for example, the term “a member” is intended to mean a single member or a combination of members, “a material”is intended to mean one or more materials, and/or the like.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one implementation, to at least one, optionally including more than one, A, with no B present (and optionally including elements other than B); in another implementation, to at least one, optionally including more than one, B, with no A present (and optionally including elements other than A); in yet another implementation, to at least one, optionally including more than one, A, and at least one, optionally including more than one, B (and optionally including other elements); etc.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

1. An apparatus, comprising:
a tissue excision device configured to excise target tissue from a lung, the tissue excision device including one or more tubular members defining an inner volume and configured to advance through lung tissue to form a tissue column within the inner volume that includes the target tissue; and
a handle assembly configured to be coupled to the tissue excision device, the handle assembly including a first actuation assembly and a second actuation assembly, the handle assembly configured to transition between a first operation mode and a second operation mode,
the first actuation assembly, when the handle assembly is in the first operation mode, configured to cause the one or more tubular members to advance through the lung tissue, seal a portion of the lung tissue, and cut the portion of the lung tissue to form the tissue column, and the second actuation assembly, when the handle assembly is in the second operation mode, configured to cause a snare to separate the tissue column from surrounding tissue.

2. The apparatus of claim 1, wherein the tissue excision device includes:
a helical coil coupled to a distal end of a tubular member of the one or more tubular members, the helical coil configured to rotate distally through tissue;
a pair of electrodes; and
a cutting device configured to cut through the lung tissue.

3. The apparatus of claim 2, wherein when the handle assembly is in the first operation mode, the first actuation assembly is configured to:
rotate the helical coil distally through the tissue toward the target tissue;
move the pair of electrodes together to clamp tissue between the pair of electrodes;
deliver energy to the lung tissue through the pair of electrodes to seal vessels in the portion of the lung tissue; and
advance the cutting device to cut the portion of the lung tissue.

4. The apparatus of claim 2, wherein the first actuation assembly includes:
a first actuator coupled to the helical coil, the first actuator including a wheel configured to rotate a shaft.

5. The apparatus of claim 4, wherein the first actuation assembly includes:
a second actuator configured to move an electrode of the pair of electrodes distally to clamp tissue between the pair of electrodes; and
a third actuator coupled to the cutting device and configured to advance the cutting device distally.

6. The apparatus of claim 5, further comprising:
a mode button configured to transition the handle assembly from the first operation mode to the second operation mode.

7. The apparatus of claim 6, wherein the mode button when actuated is configured to:
expose the snare; and
lock the third actuator to prevent the cutting device from being advanced when the handle assembly is in the second operation mode.

8. The apparatus of claim 2, further comprising:
a snare actuator configured to be moved to a first position to cause the snare to move a portion of tissue toward the pair of electrodes, the snare actuator configured to be moved to a second position to cause the snare to sever the portion of tissue from the surrounding tissue.

9. The apparatus of claim 1, further comprising:
an anchor including a distal end configured to engage the target tissue, the anchor configured to guide the tissue excision device toward the target tissue.

10. The apparatus of claim 9, wherein the handle assembly is configured to be transitioned from the first operation mode to the second operation mode after a proximal portion of the anchor engages a portion of the handle assembly indicating the target tissue is disposed in the inner volume of the one or more tubular members.

11. An apparatus, comprising:
a first actuator coupled to a helical coil of a tissue excision device, the helical coil including a first electrode on a surface thereof, the first actuator configured to rotate a shaft coupled to the helical coil to cause the helical coil to rotate distally through tissue;
a second actuator coupled to a tubular assembly including a second electrode on a distal end thereof, the second actuator configured to apply a first force to the tubular assembly to move the tubular assembly distally to clamp a portion of tissue between the first electrode and the second electrode, the first electrode and the second electrode configured to be activated with energy to seal vessels in the portion of tissue when the portion of tissue is clamped between the first electrode and the second electrode; and
a third actuator coupled to the tubular assembly, the third actuator configured to apply a second force to the tubular assembly after the portion of tissue is sealed to cause a cutting device disposed within the tubular assembly to advance through the portion of tissue to cut the portion of tissue.

12. The apparatus of claim 11, further comprising:
a mode button configured cause the tubular assembly to withdraw proximally to expose a snare near the distal end of the tubular assembly.

13. The apparatus of claim 12, wherein the portion of tissue is a first portion of tissue, the apparatus further comprising:
a snare actuator configured to be moved to a first position to cause the snare to move a second portion of tissue toward the first electrode and the second electrode, the snare actuator configured to be moved to a second position to cause the snare to sever the second portion of tissue from surrounding tissue.

14. The apparatus of claim 11, wherein the tissue is lung tissue, the apparatus further comprising:
a locking mechanism, the locking mechanism in a locked configuration configured to prevent movement of the first actuator, the locking mechanism configured to transition to a locked configuration when a distal end of the helical coil is at a predetermined depth relative to a surface of a lung.

15. The apparatus of claim 11, wherein the tubular assembly and the cutting device are connected at a proximal end, the second actuator configured to apply the first force and the third actuator configured to apply the second force to the proximal end.

16. The apparatus of claim 15, wherein the second force is greater than the first force.

17. The apparatus of claim 15, wherein a portion of the tubular assembly includes a plurality of cut outs such that the portion of the tubular assembly is compliant along a longitudinal axis thereof, the second force applied to the proximal end configured to cause the portion of the tubular assembly to compress along the longitudinal axis.

18. An apparatus, comprising
one or more actuators configured to cause one or more tubular members to advance through lung tissue, seal a portion of the lung tissue, and cut the portion of the lung tissue to form a tissue column in an inner volume defined by the one or more tubular members;
a retraction mechanism configured to cause the one or more tubular members to withdraw proximally to expose a snare near a distal end of the one or more tubular members; and
a snare actuator moveable to a first position to cause the snare to move a distal portion of the tissue column toward a first electrode and a second electrode such that the first electrode and the second electrode can seal the distal portion of the tissue column, the snare actuator further moveable from the first position to a second position to cause the snare to sever the tissue column from surrounding tissue.

19. The apparatus of claim 18, wherein the one or more actuators includes a first actuator coupled to a helical coil including a first electrode and configured to cause the helical coil to rotate distally through tissue; and
a second actuator coupled to a tubular member including a second electrode on a distal end thereof, the second actuator configured to cause the tubular member to move distally to clamp tissue between the first electrode and the second electrode.

20. The apparatus of claim 19, wherein the one or more actuators includes a third actuator coupled to a cutting device, the third actuator configured to cause the cutting device to advance through the clamped tissue to cut the clamped tissue.

21. The apparatus of claim 20, wherein the retraction mechanism is configured such that the third actuator is locked, after the snare is exposed, to prevent the cutting device from being advanced.

22. The apparatus of claim 20, wherein the second actuator and the third actuator are each a lever.

23. The apparatus of claim 20, wherein the tubular member and the cutting device are connected at a proximal end, the second actuator configured to apply a first force and the third actuator configured to apply a second force to the proximal end.

24. The apparatus of claim 23, wherein the second force is greater than the first force.

25. The apparatus of claim 23, wherein the tubular member includes a plurality of cut outs such that the tubular member is compliant along a longitudinal axis of the tubular member, the second force applied to the proximal end configured to cause the tubular member to compress along the longitudinal axis.

26. An apparatus, comprising:
a housing defining an inner volume;
an actuator disposed on the housing and coupled to a tubular member, the actuator configured to cause the tubular member to rotate distally through tissue of a lung;
a locking mechanism at least partially disposed in the housing, the locking mechanism in a locked configuration configured to prevent movement of the actuator, the locking mechanism configured to transition from an unlocked configuration to the locked configuration when a distal end of the tubular member is at a predetermined depth relative to a surface of the lung; and
a shaft configured to extend through the inner volume of the housing such that a portion of the shaft is slidably disposed through a portion of the locking mechanism, the shaft coupled to a tissue anchor configured to anchor to a target tissue site in the lung, the shaft including a groove configured to engage with the portion of the locking mechanism when the distal end of the tubular member is at the predetermined depth, to transition the locking mechanism to the locked configuration.

27. The apparatus of claim 26, wherein the tubular member is a first tubular member including a first electrode on a distal end thereof, the apparatus further comprising:
a second actuator coupled to a second tubular member including a second electrode on a distal end thereof, the second actuator configured to apply a first force to the second tubular member to move the second tubular member distally to clamp a portion of tissue between the first electrode and the second electrode, the first electrode and the second electrode configured to be activated with energy to seal vessels in the portion of tissue.

28. The apparatus of claim 27, further comprising:
a third actuator coupled to a cutting device, the third actuator when actuated configured to apply a second force to the cutting device to cause the cutting device to advance through the portion of tissue and cut the portion of tissue.

29. The apparatus of claim 27, further comprising a mode button, the mode button is configured to cause the second tubular member to withdraw proximally to expose a snare near a distal end of the second tubular member.

30. The apparatus of claim 29, wherein the portion of tissue is a first portion of tissue, the apparatus further comprising:
a snare actuator configured to be moved to a first position to cause the snare to move a second portion of tissue toward the first electrode and the second electrode, the snare actuator configured to be moved to a second position to cause the snare to sever the second portion of tissue from surrounding tissue.
